(19) United States
(12) Patent Application Publication
(10) Pub. No.: US 2026/0226511 A1
(43) Pub. Date: Aug. 6, 2026

(54) METHODS AND SYSTEMS FOR PRE-FERMENTATION TREATMENT OF ORGANIC WASTE

(71) Applicant: TRIPLEW LTD., Netanya (IL)

(72) Inventors: Rotem Tidhar, Even Yehuda (IL); Ayelet Ben Natan, Kapellen (BE); Reinout De Boeck, Merelbeke (BE); Dries Pauwels, Oostakker (BE); Fabienne Snoeck, Rijsbergen (NL)

(21) Appl. No.: 19/150,932

(22) PCT Filed: Feb. 1, 2024
(86) PCT No.: PCT/IL2024/050127

§ 371 Date: Jul. 25, 2025

Related U.S. Application Data
(60) Provisional application No. 63/482,803, filed on Feb. 2, 2023.

(51) Int. Cl. C12P 7/56

(52) U.S. Cl. CPC C12P 7/56; C12P 2201/00

(57) ABSTRACT

Methods and systems for pretreatment of organic waste prior to large-scale production of lactic acid from the organic waste are provided, which result in increased production yield. The pretreatment methods and systems of the present invention employ particular temperature and pH conditions that inhibit or even completely prevent endogenous microbial activity within the waste during the pretreatment stage. The pretreatment methods and systems of the present invention thereby reduce or even prevent unwanted utilization of reducing sugars that are naturally present within the waste, and preserve the glucose potential of the organic waste.

### FIELD OF THE INVENTION

The present invention relates to recycling of organic waste. In particular, there is provided methods and systems for treating of organic waste prior to large-scale production of lactic acid by fermentation.

### BACKGROUND OF THE INVENTION

### Lactic Acid Fermentation

Lactic acid fermentation, namely, production of lactic acid from carbohydrate sources via microbial fermentation, has been gaining interest in recent years due to the ability to use lactic acid as a building block in the manufacture of bioplastics. Lactic acid can be polymerized to form the biodegradable and recyclable polyester, polylactic acid (PLA), which is considered a potential substitute for plastics manufactured from petroleum. PLA is used in the manufacture of various products including food packaging, disposables, fibers in the textile and hygiene products industries, and more, and is the most common plastic filament utilized in 3D printing.

Production of lactic acid by fermentation bioprocesses is preferred over chemical synthesis methods for various considerations, including environmental concerns, costs and the difficulty to generate enantiomerically pure lactic acid by chemical synthesis, which is desired for most industrial applications. The conventional fermentation process is typically based on anaerobic fermentation by lactic acid-producing microorganisms, which produce lactic acid as the major metabolic end product of carbohydrate fermentation. For production of PLA, the lactic acid generated during the fermentation is separated from the fermentation broth and purified by various processes, and the purified lactic acid is then subjected to polymerization.

Lactic acid has a chiral carbon atom and therefore exists in two enantiomeric forms, D- and L-lactic acid. In order to generate PLA that is suitable for industrial applications, the D- or L-lactic acid entering the production process must be highly purified to meet the specification required for polymerization. Therefore, lactic acid bacteria that produce only L-lactate enantiomer or only D-lactate enantiomer are typically used in order to produce one discreet enantiomer (L or D).

In currently available commercial processes, the carbohydrate source for lactic acid fermentation is typically a starch-containing renewable source such as corn and cassava root. Additional sources, such as the cellulose-rich sugarcane bagasse, have also been proposed. Typically, lactic acid bacteria can utilize reducing sugars like glucose and fructose, but do not have the ability to degrade polysaccharides like starch and cellulose. Thus, to utilize such polysaccharides, the process requires adding glycolytic enzymes, typically in combination with chemical treatment, to degrade the polysaccharides and release reducing sugars, a process defined as saccharification.

Saccharification may precede the fermentation process or may be done simultaneously therewith. Saccharification before fermentation is known as separate hydrolysis and fermentation (SHF), and a process which combines saccharification and fermentation is known as simultaneous saccharification and fermentation (SSF). There are various advantages and disadvantages to each of these techniques. In SHF each of the saccharification and the fermentation stages can be conducted at its own optimal conditions (temperature, pH, etc.), whereas in SSF conditions must be applied which allow both the hydrolyzing enzymes and the fermenting organisms to operate, albeit with non-optimal functionality. On the other hand, the SSF has the advantage of requiring only one step, saving time and expenses. It has the further advantage of using-up the reducing sugars as they are released from the polysaccharides, thereby maintaining a relatively low concentration of reducing sugars and high productivity of the hydrolyzing enzymes.

An additional source of carbohydrates for lactic acid fermentation that has been proposed is complex organic waste, such as mixed food waste from municipal, industrial and commercial origin, which typically includes varied ratios of reducing sugars (glucose, fructose, lactose, etc.), starch and lignocellulosic material. Such organic waste is advantageous as it is readily available and less expensive compared to other carbohydrate sources for lactic acid fermentation. However, as such organic waste usually accumulates for a couple of days before being transferred to a waste treatment facility and/or before being subjected to the industrial fermentation process, naturally occurring microbial processes take place within the waste material before initiation of the intended industrial fermentation process. These microbial processes often involve utilizing the reducing sugars which are the key substrate of the controlled fermentation, thus compromising the efficiency of the controlled fermentation process and lowering the yield of lactic acid. The microbial processes may further produce products and side products which inhibit various stages of the fermentation process, and may also produce a different enantiomer of lactic acid than the desired enantiomer, which elevates the impurity of the lactic acid final product and may necessitate applying time-consuming and costly purification processes.

EP 1320388 discloses a method for reducing the number of viable microbial organisms and/or prions present in an organic material, said method comprising the steps of: i) providing an organic material comprising solid and/or liquid parts, ii) subjecting said organic material to the processing steps of: a) lime pressure cooking at a temperature of between 100° C. and 220° C. resulting in hydrolysis of the organic material, wherein lime is Ca(OH)₂ and/or CaO, and b) stripping ammonia from said lime pressure cooked organic material, wherein lime added in connection with stripping of ammonia and sanitation of the organic material precipitates dissolved orthophosphate, and iii) obtaining a processed organic material comprising a reduced number of viable microbial organisms and/or prions.

WO 00/02457 discloses a fermented, pasteurized preferment comprising: the fermentation product of a mixture of gluten and/or bran resulting from the hydrolysis thereof with a protease and/or lipase and/or glycosidase and/or glycanase, preferably a glucanase, followed by a fermentation with an acid forming bacterium, preferably a lactic acid forming bacterium, and a yeast, preferably in the presence of enzymes capable of liberating flavor ingredients or flavor precursors from proteins and/or carbohydrates, preferably selected from proteases and another glycosidase and/or glycanase, in particular amylases.

WO 2016/016235 discloses a process for preparing lactic acid and/or a lactate salt via fermentation of carbohydrates obtained from lignocellulosic material. In particular the process comprises: treating a lignocellulosic material with an alkaline agent comprising a caustic magnesium salt in the presence of water to provide a treated aqueous lignocellulosic material; saccharifying the treated aqueous lignocellulosic material in the presence of a hydrolytic enzyme to provide a saccharified aqueous lignocellulosic material comprising fermentable carbohydrates and a solid lignocellulosic fraction; fermenting the fermentable carbohydrates in the saccharified aqueous lignocellulosic material by means of lactic acid producing microorganism in the presence of an alkaline agent comprising a caustic magnesium salt, to provide an aqueous fermentation broth comprising a magnesium lactate; and isolating lactic acid and/or lactate salt from the fermentation broth. The saccharification and the fermentation treatments are performed as two separate steps.

There remains a need to improve the yield of lactic acid production from organic waste, and particularly to prevent unnecessary loss of substrate.

### SUMMARY OF THE INVENTION

The present invention provides methods and systems for pretreatment of organic waste prior to large-scale production of an enantiomerically pure lactic acid or a salt thereof from the organic waste, particularly L-lactic acid or a salt thereof, which result in increased production yield.

Organic waste such as mixed food waste contains a combination of free reducing sugars, such as glucose, as well as a variety of different polysaccharides such as starch, glycogen, cellulose and the like, collectively referred to as the glucose potential of the organic waste. The reducing sugars present in the organic waste are eagerly consumed by bacteria and/or other microorganisms naturally found in the organic waste, thereby diminishing the supply of reducing sugars available for the industrial fermentation process, and may produce inhibitors and other contaminants in the organic waste material. Complex organic waste, such as mixed food waste, at its raw condition may include various solids, which make it difficult to effectively sterilize the waste, and even if sterilized, it is difficult to maintain the waste under sterile conditions throughout its processing until the lactic acid production stage.

The methods and systems of the present invention advantageously addresses this limitation and inhibit or even completely prevent endogenous microbial activity within the waste during the pretreatment stage. In some embodiments, the methods and systems of the present invention inhibit or even completely prevent the loss of reducing and/or non-reducing sugars during the pretreatment stage. In particular, the methods and systems of the present invention are preferably employed on a non-sterile slurry of organic waste as soon as possible following collection of the organic waste and initial processing such as grinding and removal of plastics and inorganic solid components such as glass and sand (if present), and comprise maintaining the non-sterile organic waste slurry under particular conditions of temperature and pH until further processing. As exemplified herein below, it was surprisingly found that maintaining a non-sterile organic waste slurry at a temperature between 55-65° C. and a pH between 3.5-5.5 effectively inhibits microbial activity in the waste, as evidenced by substantially constant concentration of available glucose throughout the maintenance at the aforementioned conditions, as well as no production of gasses such as CO₂ and methane. The maintenance at the aforementioned conditions resulted in essentially complete preservation of the glucose potential of the organic waste. Thus, the methods and systems of the present invention are particularly beneficial for increasing the potential amount of lactic acid that can be produced from a given batch of organic waste and accordingly the overall yield of the production process.

The pretreatment methods and systems of the present invention further comprise according to some embodiments an improved saccharification process which advantageously can be carried out on the organic waste without first subjecting it to sterilization. The lactic acid fermentation process can only utilize reducing sugars as a substrate, and it is therefore necessary to release reducing sugars via saccharification of polysaccharides present in the waste prior to fermentation. In some embodiments, a saccharification process as disclosed herein employs heating of a non-sterile organic waste slurry to a temperature between 65-85° C. (typically between 70-75° C.) for a period of time of at least 0.25 hours (typically about 2 hours), cooling to a temperature between 45-65° C., and adding one or more saccharification enzymes after the cooling. The heating step is referred to herein as “heat treatment”. As exemplified hereinbelow, the heat treatment was surprisingly found to result in effective saccharification and conversion of substantially all the glucose potential of the organic waste into available glucose, while no notable change in available glucose, or only limited, partial conversion into glucose, was measured when saccharification enzymes were added to the non-sterile organic waste slurry before the heat treatment.

The present invention is particularly useful for food waste, which is heterogenous and typically characterized by a high level of microbial contaminants and thus susceptible to decay by natural fermentation processes during collection, transport and/or pretreatment. In some embodiments, an organic waste slurry for use with the present invention is characterized by microbial content of at least 10⁵ CFU/ml. Advantageously, even though a high level of microbial contaminants is present, maintenance of organic waste slurries at the temperature and pH conditions as disclosed herein throughout various stages of the pretreatment is effective in inhibiting microbial activity, achieving efficient saccharification and preserving the glucose potential of the waste until the controlled, large-scale, production stage. Preventing the naturally occurring microbial processes which squander the free reducing sugars, together with enhancing the saccharification of the organic waste to retrieve a maximal amount of free reducing sugars, substantially increases the yield of lactic acid that can be obtained from a batch of organic waste by fermentation.

According to a first aspect, the present invention provides a method for pretreating organic waste prior to large-scale production of lactic acid or a salt thereof from the organic waste, the method comprising:
(a) providing a non-sterile slurry of organic waste comprising non-reducing polysaccharides and reducing sugars;
(b) maintaining the non-sterile slurry of organic waste of step (a) at a temperature in the range of 55° C. to 65° C. and at a pH in the range of 3.5 to 5.5 for a first time duration of at least 1 hour, to thereby suppress endogenous microbial activity in the organic waste;
(c) raising the temperature of the non-sterile slurry of organic waste of step (b) to a temperature above the temperature of step (b) and between 65° C. to 85° C. for a second time duration of at least 0.5 hour;
(d) optionally adjusting the temperature to a temperature between 45° C. to 65° C.; and
(e) adding one or more saccharifying enzymes to the non-sterile slurry of organic waste of step (c) or step (d) for hydrolyzing polysaccharides in the organic waste to release reducing sugars, to obtain saccharified organic waste.

In some embodiments, step (c) and step (e) are performed simultaneously.

In some embodiments, the organic waste comprises plastics and/or inorganic solid components and the method comprises subjecting the organic waste to separation of said plastics and/or inorganic solid components prior to step (a).

In some embodiments, the method further comprises separating the saccharified organic waste into a liquid phase comprising reducing sugars and a solid phase, and subjecting the liquid phase to sterilization.

According to another aspect, the present invention provides a method for pretreating organic waste prior to large-scale production of lactic acid or a salt thereof from the organic waste, the method comprising:
(a) providing a non-sterile slurry of organic waste comprising non-reducing polysaccharides and reducing sugars;
(b) maintaining the non-sterile slurry of organic waste of step (a) at a temperature in the range of 55° C. to 65° C. and at a pH in the range of 3.5 to 5.5 for a first time duration of at least 1 hour, to thereby suppress endogenous microbial activity in the organic waste;
(c) raising the temperature of the non-sterile slurry of organic waste of step (b) to a temperature above the temperature of step (b) and between 65° C. to 85°° C. for a second time duration of at least 0.5 hour;
(d) adjusting the temperature to a temperature between 45° C. to 65° C. and adding one or more saccharifying enzymes for hydrolyzing polysaccharides in the organic waste to release reducing sugars, to obtain saccharified organic waste; and
(e) separating the saccharified organic waste into a liquid phase comprising reducing sugars and a solid phase, and subjecting the liquid phase to sterilization, thereby pretreating the organic waste to obtain a feedstock material for large-scale production of lactic acid or a salt thereof.

In some embodiments, the one or more saccharifying enzymes comprise at least one of: a glucoamylase, an alpha-amylase and a pullulanase.

In some embodiments, the second time duration of step (c) is in the range of 0.5 hours to 5 hours.

According to a further aspect, the present invention provides a method for pretreating organic waste prior to large-scale production of lactic acid or a salt thereof from the organic waste, the method comprising:
(A) providing a non-sterile slurry of organic waste comprising non-reducing polysaccharides and reducing sugars;
(B) heating the non-sterile slurry of organic waste to a temperature between 65° C. to 85° C. for a time duration of at least 1 hour;
(C) optionally adjusting the temperature to a temperature between 45° C. to 65° C.; and
(D) adding one or more saccharifying enzymes to the non-sterile slurry of organic waste of step (B) or step (C) for hydrolyzing polysaccharides in the organic waste to release reducing sugars, to obtain saccharified organic waste.

In some embodiments, steps (B) and (D) are carried out simultaneously.

According to a further aspect, the present invention provides a method for suppressing endogenous microbial activity in an organic waste material prior to subjecting the waste material to controlled large-scale production of lactic acid or a salt thereof, the method comprising the steps of:
(a) providing a non-sterile slurry of organic waste material comprising at least one of non-reducing polysaccharides and reducing sugars; and
(b) maintaining the waste material of step (a) at a temperature in the range of 55° C. to 65° C. and at a pH in the range of 3.5 to 5.5 for a first time duration of at least 1 hour, to thereby suppress endogenous microbial activity in the waste material prior to subjecting the waste material to controlled large-scale production of lactic acid or a salt thereof.

In some embodiments, the organic waste comprises plastics and/or inorganic solid components and the method comprises subjecting the organic waste to separation of said plastics and/or inorganic solid components prior to step (a).

In some embodiments, step (b) is performed at a pH in the range of 4 to 5.

In some embodiments, the first time duration of step (b) is in the range of 1 to 96 hours. In additional embodiments, the first time duration of step (b) is in the range of 12 to 48 hours.

In some embodiments, the method further comprises applying mechanical treatment to the waste material prior to, simultaneously with, and/or subsequently to step (b). In some embodiments, the mechanical treatment is selected from the group consisting of grinding, chipping, shredding, milling, stirring, and a combination thereof.

In some embodiments, the waste material is selected from the group consisting of food waste, municipal waste, agricultural waste, plant material, and a combination thereof.

In some embodiments, the non-sterile slurry of organic waste contains microbes at a concentration of at least 10⁵ CFU/ml. In additional embodiments, the non-sterile slurry of organic waste contains microbes at a concentration of at least 10⁷ CFU/ml.

According to another aspect, the present invention provides a method for producing lactic acid from organic waste, the method comprising the steps of:
(i) subjecting the organic waste to pretreatment comprising a pretreatment method according to the methods disclosed herein; and
(ii) adding a lactic acid producing microorganism to the pretreated organic waste and incubating in a fermentation reactor under controlled conditions for lactic acid production by the lactic acid producing microorganism, to thereby produce lactic acid. Other objects, features and advantages of the present invention will become clear from the following description, examples and drawings.

### BRIEF DESCRIPTION OF THE FIGURES

FIG. 1 shows a block diagram of a method for waste material pretreatment and lactic acid production enhancement, according to certain embodiments of the present invention.

FIG. 2 shows a graph representing maintaining waste material at temperature and pH conditions according to the present invention for 16 hours.

FIG. 3 shows a graph representing maintaining waste material at 60° C. and pH 4.5 for 18 hours with a first dose of saccharifying enzymes, followed by heat treatment at 70° C. for 2 hours, and then reducing temperature to 60° C. and adding a second dose of saccharifying enzymes.

### DETAILED DESCRIPTION OF THE INVENTION

The present invention provides methods and systems for preserving glucose potential of an organic waste material prior to subjecting the waste material to controlled lactic acid fermentation. The methods and systems of the present invention are useful for increasing the yield of lactic acid in the controlled fermentation process, e.g., by suppressing unwanted substrate consumption by endogenous microorganisms present in the organic waste and by further preventing formation of inhibitory side products and other impurities within the waste material. The methods and systems of the present invention involve subjecting a provided waste material to particular temperature and pH conditions that inhibit microbial activity, and maintaining the waste material under these conditions until further processing.

The methods and systems of the present invention are employed on a non-sterile slurry of organic waste, preferably as soon as possible following collection of the organic waste and initial processing such as grinding and removal of plastics and inorganic solid components (if present).

As used herein, a “slurry” of organic waste refers to a mixture of the organic waste and water, typically containing solid particles of the organic waste. A slurry of organic waste as used herein is typically formed by collecting waste material from various sources; subjecting the waste material to separation of plastics and inorganic solid components such as glass, metal and sand, to remove most and preferably all of the plastics and inorganic solid components; reducing the particle size of the waste material, e.g., by shredding or grinding; adding water if necessary; and creating a suspension of organic waste material in the water. In some embodiments, forming a slurry of organic waste according to the present invention, particularly a slurry of food waste, comprises subjecting the waste to depackaging, namely, removal of packaging material, including plastic, metal and glass packaging material. In some embodiments, the organic waste may naturally be in the form of a slurry.

An organic waste slurry according to the present invention (e.g. a food waste slurry) is characterized by a solid content (dry matter content) in the range of 5-50% (namely, characterized by a liquid or moisture content in the range of 50-95%), including each value within the range. In some embodiments, an organic waste slurry according to the present invention is characterized by a solid content in the range of 10-30% (namely, a liquid or moisture content in the range of 70%-90%), including each value within the range. In some embodiments, an organic waste slurry according to the present invention is characterized by a solid content in the range of 15-35% (namely, a liquid or moisture content in the range of 65%-85%) including each value within the range.

In some embodiments, an organic waste slurry according to the present invention is characterized by a water content in the range of 50-95%, including each value within the range. In some embodiments, an organic waste slurry according to the present invention is characterized by a water content in the range of 70%-90%, including each value within the range. In some embodiments, an organic waste slurry according to the present invention is characterized by a water content in the range of 65%-85%, including each value within the range.

In some embodiments, the organic waste for use with the present invention (e.g. food waste) naturally contains the aforementioned solid/liquid/water content. In other embodiments, water is added to the organic waste (e.g., food waste) to obtain a slurry characterized by the aforementioned solid/liquid/water content.

An organic waste slurry according to the present invention (e.g. a food waste slurry) is pumpable and mixable, and thus suitable for further handling and processing according to the present invention.

In some embodiments, the waste material is maintained under the temperature and pH conditions that inhibit microbial activity until the waste material is subjected to saccharification (preferably heat treatment followed by saccharification, as disclosed herein). In additional embodiments, the waste material is maintained under these conditions between saccharification and the controlled lactic acid fermentation.

The maintenance under the temperature and pH conditions that inhibit microbial activity may optionally begin from any point in the process of waste management, following the extraction of the waste material from its source of origin. Several processes may be applied to the waste material while it is maintained under the particular conditions disclosed herein, the processes including mechanical treatment, solid-liquid separation, saccharification, or any other relevant waste treatment process. Alternatively, when a certain waste treatment process requires temperature and/or pH which are different from the particular conditions disclosed herein (for example, temperature and/or pH that are required for the activity of a saccharifying enzyme), the temperature and/or pH may be adjusted to those required for the treatment process, and may be adjusted back to those disclosed herein after the treatment process is completed.

As used herein, the term “lactic acid” refers to the hydroxycarboxylic acid with the chemical formula CH₃CH(OH)CO₂H. The terms lactic acid or lactate (unprotonated lactic acid) can refer to the stereoisomers of lactic acid: L-lactic acid/L-lactate, D-lactic acid/D-lactate, or to a combination thereof.

As used herein, the terms “reducing sugar(s)”, “free sugar(s)” and “available sugar(s)” are used interchangeably and refer to soluble sugar molecules which can be utilized in their current state as a substrate for lactic acid fermentation. The reducing sugars typically comprise C5 sugars (pentoses), C6 sugars (hexoses) or a combination thereof. In some embodiments, the reducing sugars comprise glucose. In some embodiments, the reducing sugars comprise xylose.

As used herein, the terms “non-reducing carbohydrate(s)”, “polysaccharide(s)” and “non-reducing sugar(s)” are used interchangeably, and refer to polymeric sugar molecules which cannot be utilized in their current state for lactic acid fermentation and require saccharification/hydrolysis in order to become available for fermentation. Examples include starch, cellulose, hemicellulose, and combinations thereof.

As used herein, the term “maintaining at a temperature/pH in a range of X for a time duration” refers to maintaining the temperature/pH anywhere within the defined range for at least 70%, at least 75%, at least 80%, at least 85%, at least 90%, at least 95%, at least 97%, at least 98%, at least 99%, at least 99.5% or at least 99.9% of the time duration, allowing for possible deviations of the temperature/pH from the defined range.

For most industrial applications, L-lactic acid monomers with high purity are required in order to produce PLA with suitable properties. Thus, the methods and systems of the present invention are directed, in particular, to processes for the production of L-lactate salts at high yields, which can then be converted to L-lactic acid suitable for industrial use.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

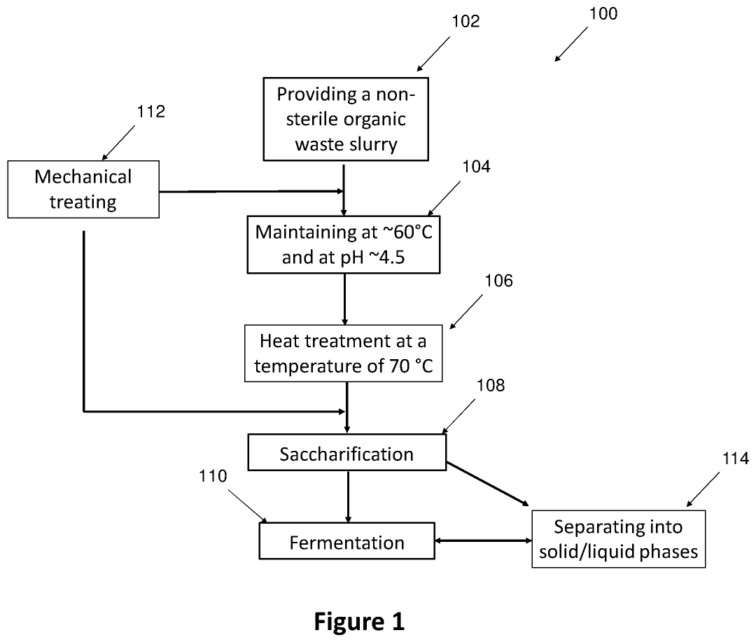

Referring now to the drawings, FIG. 1 is a block diagram of a process, referenced 100, for waste material pretreatment followed by industrial lactic acid production by fermentation, according to some embodiments of the present invention. According to the illustrated embodiments, the pretreatment process comprises maintaining a non-sterile organic waste slurry at a temperature in the range of 55-65° C. (e.g., at a temperature of about 60° C.) and a pH between 3.5-5.5 (e.g., at a pH of about 4.5) for a first period of time (e.g., for 1-96 hours), followed by a heat treatment comprising heating of the non-sterile organic waste slurry to a temperature between 65-85° C. (e.g., to 70° C.) for a second period of time (e.g., for 15 minutes-3 hours) and saccharification, with or without cooling prior to the saccharification, as will be further detailed below. The process also includes solid-liquid separation and mechanical treatment which may be applied at various time points during the process, as will be detailed below.

Step 102 includes providing an organic waste material, typically an organic waste material in the form of a slurry. An organic waste for use with the methods and systems disclosed herein was not subjected to sterilization.

Organic waste suitable for use according to the present invention is typically a complex, heterogenous, organic waste comprising solid and non-solid materials. A complex, heterogenous, organic waste includes carbohydrates for fermentation (soluble carbohydrates available for fermentation and/or polysaccharides that need to be decomposed via enzymes to release soluble carbohydrates for fermentation) and further contains impurities such as salts, fats, oils, lipids, proteins, color components, and/or inert materials and more. Organic waste for use with the present invention may also comprise inorganic solid components such as plastics, glass and the like. Organic waste for use with the present invention comprises endogenous microorganisms. The organic waste material may be a product of municipal waste, food waste, agricultural waste and/or plant matter.

Reducing sugar is the substrate that needs to be preserved for the controlled fermentation process. In some embodiments of the present invention the provided waste material includes water and/or other aqueous and/or liquid solutions. In additional embodiments of the invention water or other aqueous and/or liquid solutions may be added to the waste material throughout process 100, according to need, once or several times, intermittently and/or continuously. Each possibility represents a separate embodiment.

In some particular embodiments, the organic waste for use with the present invention is food waste.

Food waste in accordance with the present invention encompasses food waste and beverages of plant origin and/or animal origin. Food waste in accordance with the present invention encompasses household food waste, commercial food waste, and/or industrial food waste. Each possibility represents a separate embodiment. The organic food waste may originate from vegetable and fruit residues, plants, cooked food, protein residues, slaughter waste, and/or combinations thereof. Each possibility represents a separate embodiment. Industrial organic food waste may include factory waste such as by products, factory rejects, market returns or trimmings of inedible food portions (such as peels). Commercial organic food waste may include waste from shopping malls, restaurants, supermarkets, etc.

Food waste according to the present invention is typically mixed food waste, comprising one or more of: bakery waste, dairy waste, animal-origin food waste including meat, poultry and fish waste, fruit and vegetable waste, and grain-based food waste (e.g., rice, couscous, pasta, noodles). In some embodiments, mixed food waste according to the present invention comprises a combination of food wastes selected from bakery waste, dairy waste, animal-origin food waste including meat, poultry and fish waste, fruit and vegetable waste, and grain-based food waste (e.g., rice, couscous, pasta, noodles).

Food waste typically comprises solid components originating from food products or residues, e.g., food particles and debris, bones and bone fragments, shells and shell fragments, seeds and seed fragments, peels and the like, and also solids that do not originate from food products or residues, e.g., plastics, glass and metals, originating, for example, from packaging material. In some embodiments, pretreatment according to the present invention is carried out on a slurry of food waste after it was subjected to depackaging, to remove most or even all of the packaging material.

Plant material in accordance with the present invention encompasses agricultural waste and manmade products such as paper waste.

The waste material may be provided to a designated waste treatment facility or may be provided to a temporary collection point. The temporary collection point may be stationary, e.g., an underground sump or a storage house, or may include a waste transporting means, such as a truck, a boat, and the like.

According to some embodiments of the invention, the waste does not undergo any treatment prior to being subjected to pretreatment process 100, particularly does not undergo sterilization or pasteurization. In other embodiments, the waste undergoes separation of plastics and inorganic solid components and grinding to form a slurry. According to some embodiments, the slurry of waste material includes at least 10⁵ CFU/ml (Colony Forming Units) of endogenous microbes (i.e., bacteria, yeast, fungi, and the like). According to some further embodiments, the slurry of waste material includes at least 10⁷ CFU/ml. It is noted that the present invention is particularly beneficial for waste streams which contain viable microbes, which, when not treated, can contaminate the waste and exploit the beneficial compounds therein in their metabolic processes.

Step 104 includes maintaining the waste material at a temperature in the range of 55-65° C., and at a pH in the range of 3.5-5.5, including each value within the specified ranges for a first time duration. In some embodiments, the waste material is maintained at a temperature in the range of 50-85°° C., including each value within the specified range. In additional embodiments, the waste material is maintained at a temperature in the range of 55-85° C., including each value within the specified range. In yet additional embodiments, the waste material is maintained at a temperature in the range of 55-75° C., including each value within the specified range. In yet additional embodiments, the waste material is maintained at a temperature in the range of 55-70° C., including each value within the specified range. In one embodiment, the temperature is in the range of 57-62° C., including each value within the specified range. In another embodiment, the temperature is about 60° C. In some particular embodiments, the temperature is 60° C.

In some embodiments, the pH is in the range of 4-5.5, including each value within the specified range. In one embodiment, the pH is in the range of 4-5, including each value within the specified range. In another embodiment, the pH is in the range of 4.2-4.6, including each value within the specified range.

The waste material is typically maintained under these conditions for at least 0.5 hour, preferably for at least 1 hour. In some embodiments, the waste material is maintained under these conditions for 1-96 hours. Thus, in one embodiment, the first time duration is at least 0.5 hour, preferably at least 1 hour. In additional embodiments, the first time duration is in the range of 1 to 96 hours, including each value within the specified range. In another embodiment, the first time duration is in the range of 1 to 10 hours, including each value within the specified range. In additional embodiments, the first time duration is in the range of 1 to 5 hours, including each value within the specified range. In another embodiment, the first time duration is in the range of 12 to 48 hours, including each value within the specified range. In additional embodiments, the first time duration is in the range of 10-20 hours, including each value within the specified range. In some embodiments, the waste material is kept under the particular temperature and pH conditions up to 4 days prior to undergoing additional treatments and/or controlled fermentation. In other embodiments, the waste material undergoes additional treatments while being maintained in the particular temperature and pH conditions, e.g., mechanical treatment, solid-liquid separation, introduction of saccharifying enzymes, as will be described further with reference to steps 108, 112, and 114.

Maintaining the waste material under the conditions disclosed herein is considered conducive to lactic acid production via controlled fermentation from several perspectives. First, the temperature and pH conditions disclosed herein suppress activity of endogenous microorganisms, i.e., microorganisms which are naturally found within the waste material, or which are found in the ambient surroundings and contaminates the waste material as it is transferred from one location to another. In waste material which is not kept under conditions that inhibit microbial activity, the endogenous microorganisms feed on the free reducing sugars in the waste material to produce a variety of products, such as D- and L-lactic acid, pyruvate, succinic acid, acetic acid, formic acid, and ethyl alcohol. Many of the products of the naturally occurring microbial reactions are detrimental to controlled lactic acid production. In particular, some of the compounds serve as inhibitors and toxins to the enzymes and bacteria that are introduced into the waste material at different stages of the controlled fermentation process. In addition, reducing sugar, which is the substrate for the lactic acid production, is consumed. Finally, the non-regulated microbial reactions may induce racemization of lactic acid, necessitating a time-consuming and costly purification of the eventual fermentation product. Maintaining the waste material under the conditions disclosed herein was found to have a highly effective suppressing effect on microbial activity.

Second, the relatively elevated temperature in which the waste material is maintained according to the present invention, as compared with naturally occurring surrounding temperature or with cooling conditions, promotes liquification of the waste material. Liquification is the process by which solid-state matter becomes liquid, either by melting or by dissolving into water or any other solvent. As parts of the solid matter in the waste material merge with the liquid phase thereof, the components which are beneficial for the controlled fermentation become more readily accessible to enzymes and/or microbes which are utilized for different stages of the lactic acid production, improving the efficiency and yield of the process. Even when not actually liquifying the solid waste matter, the pH and temperature conditions disclosed herein contribute to softening and increasing fluidity of strong and harsh fibers, which may otherwise damage or jam the waste handling equipment.

Step 106 includes raising the temperature of the waste material to a temperature above 65° C., for a second time duration (this step is referenced herein as “heat treatment”). The temperature may be in the range of 65-85° C., for example 65-80° C., 70-85° C., 70-80° C. or 70-75° C., including any value within the specified ranges. Each possibility represents a separate embodiment. In one embodiment, the temperature is in the range of 65-85° C., including each value within the specified range. In a further embodiment, the temperature is in the range of 68-73° C., including each value within the specified range. In some embodiments, the second time duration is in the range of 15 minutes-5 hours, including each value within the specified range. In additional embodiments, the second time duration is in the range of 15 minutes-3 hours, including each value within the specified range. In one embodiment, the second time duration is in the range of 15 minutes-2.5 hours, including each value within the specified range. In some embodiments, the second time duration is between 0.5-5 hours, including each value within the specified range. In additional embodiments, the second time duration is between 0.5-3 hours, including each value within the specified range. In some embodiments, the second time duration is between 0.5-2.5 hours, including each value within the specified range. The pH of the waste material during the heat treatment is usually maintained within the range of 3.5 to 5.5, including each value within the specified range. In general, the required time duration is inversely correlated to the temperature, i.e., the higher the temperature the shorter the required time duration. The time duration of the step 106 may exceed 5 hours. Step 106 is generally conducted as a preparatory stage to the saccharification procedure, as described herein in step 108. Although saccharification is generally performed at a temperature in the range of 50-60° C., it has surprisingly been discovered that subjecting the waste material to heat treatment prior to saccharification substantially improved the efficiency of the saccharification, significantly increasing the amount of reducing sugar released from the non-reducing polysaccharides. Also surprisingly, it was shown that a relatively mild increase in the temperature of the waste material by a mere 10° C. to a temperature of about 70° C., provided a significant enhancement in the yield of reducing sugar.

Step 108 includes saccharifying the non-reducing polysaccharides in the waste material to release reducing sugars. Saccharification according to the present invention generally includes adding one or more saccharide-degrading enzymes (also termed saccharifying enzymes or saccharification enzymes), typically glycolytic enzymes which hydrolyze the glycosidic bonds of the saccharides in the waste material, to release reducing sugars for fermentation. The saccharides include bi-saccharides (di-saccharides), oligosaccharides, polysaccharides and glycoconjugates. Saccharide-degrading enzymes may be selected from the group consisting of glycoside hydrolases, polysaccharide lyases and carbohydrate esterases. Each possibility represents a separate embodiment. In some embodiments, the saccharide-degrading enzymes may be modified enzymes (i.e., enzymes that have been modified and are different from their corresponding wild-type enzymes). In some embodiments, the modification may include one or more mutations that result in improved activity of the enzyme. In some embodiments, the saccharide-degrading enzymes are wild type (WT) enzymes.

The broad group of saccharide-degrading enzymes is divided into enzyme classes and further into enzyme families according to a standard classification system (Cantarel et al. 2009 Nucleic Acids Res 37: D233-238). An informative and updated classification of such enzymes is available on the Carbohydrate-Active Enzymes (CAZy) server (www.cazy.org).

In some embodiments, the saccharide-degrading enzymes are selected from amylases, cellulases and hemicellulases. Each possibility represents a separate embodiment of the present invention.

A cellulase may be selected from, but not limited to: endo-(1,4)-D-glucanase, εχo-(1,4)-β-\ddot{υ}-glucanase, β-glucosidases, Carboxymethylcellulase (CMCase); endoglucanase; cellobiohydrolase; avicelase, celludextrinase, cellulase A, cellulosin AP, alkali cellulase, and pancellase SS. Each possibility is a separate embodiment.

A hemicellulase may be a xylanase. Non-limiting examples of additional hemicellulases include arabinofuranosidases, acetyl esterases, mannanases, a-D-glucuronidases, β-xylosidases, β-mannosidases, β-glucosidases, acetyl-mannanesterases, a-galactosidases, -a-Larabinanases, and β-galactosidases. Each possibility represents a separate embodiment of the present invention.

An amylase may be selected from, but not limited to: glucoamylase, a-amylase; (1,4-a-D-glucan glucanohydrolase; glycogenase) β-Amylase; (1,4-a-D-glucan maltohydrolase; glycogenase; saccharogen amylase) γ-Amylase; (Glucan 1,4-a-glucosidase; amyloglucosidase; Exo-1,4-a-glucosidase; lysosomal a-glucosidase; 1,4-a-D-glucan glucohydrolase) and pullulanase (limit dextrinase; amylopectin 6-glucanohydrolase; bacterial debranching enzyme; debranching enzyme; alpha-dextrin endo-1,6-alpha-glucosidase; R-enzyme; pullulan alpha-1,6-glucanohydrolase). Each possibility is a separate embodiment.

In some embodiments, the saccharide-degrading enzymes used in the present invention are disaccharide-degrading enzymes. In some embodiments, the disaccharide-degrading enzymes are selected from lactases and invertases. Each possibility represents a separate embodiment of the present invention.

The saccharide-degrading enzymes according to the present invention may be from a bacterial source. In some embodiments, the bacterial source is a thermophilic bacterium. The term “thermophilic bacterium” as used herein indicates a bacterium that thrives at temperatures higher than about 45° C., preferably above 50° C. Typically, thermophilic bacteria according to the present invention have optimum growth temperature of between about 45°° C. to about 75° C., preferably about 50-70° C. Non-limiting examples of thermophilic bacterial sources for saccharide-degrading enzymes include: Cellulases and hemicellulases—Clostridium sp. (e.g. Clostridium thermocellum), Paenibacillus sp., Thermobifida fusca; Amylases-Bacillus sp. (e.g. Bacillus stearothermophilus), Geobacillus sp. (e.g. Geobacillus thermoleovorans), Chromohalobacter sp., Rhodothermus marinus.Each possibility is a separate embodiment.

In additional embodiments, the bacterial source of the saccharide-degrading enzymes is a mesophilic bacterium. The term “mesophilic bacterium” as used herein indicates a bacterium that thrives at temperatures between about 20°° C. and 45° C. Non-limiting examples of mesophilic bacterial sources for saccharide-degrading enzymes include: Cellulases and hemicellulases—Klebsiella sp. (e.g. Klebsiella pneumonia), Cohnel sp., Streptomyces sp, Acetivibrio cellulolyticus, Ruminococcus albus; Amylases-Bacillus sp. (e.g. Bacillus amyloliquefaciens, Bacillus subtilis, Bacillus licheniformis), Lactobacillus fermentum. A person of skill in the art understands that some mesophilic bacteria (e.g., several Bacillus sp.) produce thermostable enzymes.

The saccharide-degrading enzymes according to the present invention may also be from a fungal source. Non-limiting examples of fungal sources for saccharide-degrading enzymes include: Cellulases and hemicellulases—Trichoderma reesei, Humicola insolens, Fusarium oxysporum; Amylases (e.g., glucoamylases)—Aspergillus niger Aspergillus oryzae, Penicillium fellutanum, Thermomyces lanuginosu.

Additional sources for saccharide-degrading enzymes for use in accordance with the present invention can be found, for example, at the CAZy server mentioned above.

According to some embodiments of the present invention, the saccharifying enzymes may be added at a concentration sufficient for saccharification of at least 75%, at least 85%, at least 90%, at least 95%, at least 98%, at least 99%, or at least 99.9% of the polysaccharides in the waste material.

In one embodiment of the present invention, step 108 (i.e., saccharification) is performed at the same temperature as the heat treatment, and optionally concurrently therewith. When saccharification is carried out concurrently with the heat treatment, the second time duration is typically extended to a time duration needed for sufficient or complete saccharification of polysaccharides in the waste material. In another embodiment, the temperature of the waste material may be adjusted prior to adding the saccharifying enzymes, typically to a temperature in the range of 45-65° C., including each value within the specified range. In some embodiments, the saccharification is carried out at the temperature range disclosed herein as efficient in inhibiting microbial activity within the waste material, between 55-65° C. In one embodiment, the temperature of the waste material during the saccharification is in the range of 50-62° C., including each value within the specified range. In another preferable embodiment, the saccharification is performed at a temperature in the range of 58-62° C., including each value within the specified range. The pH of the waste material during the saccharification is usually maintained within the range of 3.5 to 5.5, including each value within the specified range. Use of the temperature and pH conditions disclosed herein during the saccharification step confers the advantages of suppressing activity of endogenous bacteria, liquifying the waste material, etc., also during saccharification.

In general, the temperature and the pH of the waste material during saccharification need to be controlled such that the utilized enzymes are capable of effectively hydrolyzing the non-reducing carbohydrates. It is to be understood that when the saccharification is carried out under the temperature and pH conditions that inhibit microbial activity, the saccharide-degrading enzyme(s) is an enzyme that is active in these conditions.

The saccharification step may be carried out for a period of time between 0.25-24 hours, typically between 1-24 hours, 1.5-24 hours, 5-24 hours, 8-24 hours, 1-15 hours or 1-10 hours, including each value within the specified ranges. Each possibility represents a separate embodiment of the present invention.

A combination of a variety of saccharifying enzymes may be applied, suited for saccharifying a variety of types of saccharides, e.g., cellulose, glucan, starch, and the like. In one embodiment of the present invention, one of the factors which controls the types and relative concentrations of enzymes used in the saccharification process may be the relative prevalence of the different types of polysaccharides in the waste material. For example, when treating a batch of waste material known to originate from crop residue, enzymes from the cellulase group may be used predominantly, to saccharify the high concentrations of cellulose. When the waste material contains high levels of starch, e.g., in waste coming from bakeries or cafeterias, the saccharifying enzymes may include a substantial amount of amylase. Alternatively, other considerations may affect the types of utilized saccharifying enzymes, such as cost-effectiveness, yield, manufacturer-recommended conditions of the various enzymes, and the like.

In some embodiments of the present invention, the sequential order of steps 104, 106 and 108 may be altered. For example, step 106 may precede step 104, such that the provided waste material of step 102 is directly subjected to the heat treatment of step 106. Following the heat treatment, the waste material may be either subjected to saccharification (i.e., step 108), or may be maintained at the temperature and pH conditions that inhibit microbial activity (i.e., step 104) prior to undergoing saccharification. In effect, the waste material may be kept under the conditions that inhibit microbial activity prior to the heat treatment, between the heat treatment and saccharification, and/or following saccharification prior to the fermentation of step 110. Each possibility represents a separate embodiment.

Step 110 includes controlled production of lactic acid via fermentation of the reducing sugars of the waste material. “Controlled production” or “controlled fermentation” as used herein refers to lactic acid fermentation that is carried out in a fermenter under controlled conditions, for example: lactic acid fermentation under the control of one or more of the following parameters: temperature, pH, levels of nutrients, agitation rate and aeration (aerobic/anaerobic/microaerophilic conditions). Typically, separation of solid components (step 114, as will be detailed below) and/or sterilization are carried out prior to step 110. Following the controlled production stage, the fermentation broth is processed to recover the fermentation product, namely, lactic acid or a salt thereof.

Lactic acid fermentation is performed using a lactic acid-producing microorganism. “LA-producing microorganisms” as used herein refers to microorganisms that produce lactic acid as the major metabolic end product of carbohydrate fermentation. Currently preferred is the use of microorganisms which produce only L-lactic acid. The LA-producing microorganisms may naturally produce only L-lactic acid, or may be genetically modified to produce only L-lactic acid, for example by knocking out one or more enzymes involved in the synthesis of the undesired D-enantiomer. LA-producing microorganisms include various bacteria, including for example Lactobacillus species and Bacillus species, fungi, and yeast. Each possibility represents a separate embodiment.

The saccharification and lactic acid fermentation, i.e., steps 108 and 110, may be carried out simultaneously (simultaneous saccharification and fermentation, SSF). Alternatively, enzymatic digestion of polysaccharides may be carried out before lactic acid fermentation, in the same reactor in which fermentation is carried out or in a different reactor (separate hydrolysis and fermentation, SHF). Each possibility (SSF, SHF) represents a separate embodiment of the present invention.

Fermentation is typically performed in the presence of an alkaline compound, such as a metal oxide, a carbonate or a hydroxide as detailed above. Suitable alkaline compounds include, but are not limited to, MgO, CaO, CaCO₃, MgCO₃, NaOH, KOH, NH₄OH, Ca(OH)₂, Mg(OH)₂, and a mixture or combination thereof. Each possibility represents a separate embodiment. The alkaline compound is added to adjust the pH of the fermentation broth to a desired value, typically in the range of 5 to 7, including each value within the specified range. The alkaline compound further results in the neutralization of the L-lactic acid to a lactate salt. During fermentation, the pH in the fermenter decreases due to the production of the lactic acid, which adversely affects the productivity of the lactic acid-producing microorganism. Adding bases such as magnesium-, sodium-, potassium-, or calcium-hydroxide adjusts the pH by neutralizing the lactic acid thereby resulting in the formation of a lactate salt.

Typically, the fermenting is carried out under anaerobic or microaerophilic conditions, using batch, fed-batch, continuous or semi-continuous fermentation. Each possibility represents a separate embodiment of the present invention.

In batch fermentation, the carbon substrates and other components are loaded into the reactor, and when the fermentation is completed, the product is collected. Except for the alkaline compound discussed above for pH control, other ingredients are not added to the reaction before it is completed. The inoculum size is typically about 5-10% of the liquid volume in the reactor. The fermentation is kept at substantially constant temperature and pH, where the pH is maintained by adding the alkaline compound.

In fed-batch fermentation, the substrate is fed continuously or sequentially to the reactor without the removal of fermentation broth (i.e., the product(s) remain in the reactor until the end of the run). Common feeding methods include intermittent, constant, pulse-feeding, and exponential feeding. Each possibility represents a separate embodiment.

In continuous fermentation, the substrate is added to the reactor continuously at a fixed rate, and the fermentation products are taken out continuously.

In semi-continuous processes, a portion of the culture is withdrawn at intervals and fresh medium is added to the system. Repeated fed-batch culture, which can be maintained indefinitely, is also considered a semi-continuous process.

Lactic acid fermentation is typically carried out for about 1-4 days or any amount therebetween, for example, 1-2 days, or 2-4 days, or 3-4 days, including each value within the specified ranges.

The waste material subjected to lactic acid production process 100 is often of a large volume and includes a non-uniform mixture of a variety of liquids and solids. Therefore, potentially beneficial compounds in the waste material are not always accessible to treatment reagents, e.g., reactive compounds, enzymes, and the like, and so do not contribute to lactic acid production. In addition, some of the macromolecules in the waste material are degraded through surface erosion, gradually proceeding via an erosion front or a plurality of erosion fronts to the bulk, which can be slow and not always effective. Referring now to step 112, the waste material is subjected to mechanical treatment for increasing the surface area and/or for improving the turnover of the organic components and the active ingredients within the waste material. The mechanical treatment may include, for example, grinding, chipping, shredding, mincing and/or milling.

The mechanical treatment may also include stirring the waste material or a portion thereof. The stirring is intended to thoroughly mix the waste material, allowing interaction of all parts of the waste material with the treatment reagents. The mixing may also enhance the effectivity of processes such as heating, aerating and/or depressurizing applied to the waste material. Stirring may be at a rate in the range of 10 to 1000 RPM, e.g., in the range of 30-100 RPM, 50-200 RPM, 150-500 RPM, 300-700 RPM, or 500-1000 RPM, including any value within the specified ranges. The rate of stirring, or any other parameter, such as power, force, directionality, means of stirring (e.g., internal or external), and the like, may be substantially fixed throughout process 100, or may be adjusted, and/or activated and deactivated, at different steps. For example, the rate and/or force of the stirring may be highest during step 104, when the waste material is at a relatively raw state, and may be reduced as the process advances, in correlation with liquification of the waste material. Alternatively, the stirring may begin in a mild manner, e.g., at a relatively low stirring rate, and may intensify as treatment agents are added into the waste material, for example in steps 108 and 110, so as to accelerate the treatment procedures. The rate and power of the stirring may be increased in accord, or may optionally be reverse correlated, such that when the rate is increased the power is reduced. Each possibility represents a separate embodiment. Any other combination of stirring parameters during the various steps may be utilized. Some variables which may determine the selected stirring parameters include, but are not limited to, gas-liquid mass transfer, feed distribution, local oxygen concentration, shear rate distribution, and local mixing intensity,

Any embodiment of mechanical treatment as described in step 112 may be performed at any one or more of the various stages of process 100, or throughout the entire process. By way of unlimiting example, the waste material may undergo grinding, chipping, etc., proximately to when the waste material is provided (i.e., step 102), before being submitted to the conditions that inhibit microbial activity (i.e., step 104), simultaneously therewith, and/or subsequently to step 104. Alternatively or additionally, the waste material may be mechanically treated to enlarge the surface area of the polysaccharide compounds prior to commencement of saccharification (i.e., step 108), and/or simultaneously therewith. In another embodiment, the waste material may be crudely ground, chipped, shredded, milled, or otherwise broken up, at an early stage of the pre-treatment (e.g., before applying the conditions that inhibit microbial activity), and more finely ground, chipped, etc., at a later stage of the process (e.g., before saccharification and/or fermentation). Mechanical treatment in the form of stirring may also be applied continuously, or at selected stages of process 100.

The organic waste material according to the present invention typically contains solid components, including organic and inorganic solid components. Although the pretreatment induces liquifying of the waste material, there may remain solid particles of various shapes, size and texture within the waste material. The solid particles often cannot be effectively treated to produce reducing sugars for the controlled fermentation. The solid particles may interfere with the treatment processes to which the waste material is subjected, clogging up machinery and/or hampering access of treatment reagents to productive waste materials. Referring now to step 114, a solid-liquid separation is applied to the waste material. Solid-liquid separation may include, for example, filtration, decantation, and the like. The waste material or portions thereof may be processed by a screw press, a filter press, may be centrifuged, and/or may be processed by any other liquid-solid separating method known in the art or a combination of methods. Following the separation, the liquid phase usually includes the free reducing sugars and/or saccharifyable carbohydrates and is collected for continued processing.

The solid phase may also be independently collected to be selectively treated. Methods for breaking-up or molding materials, such as mechanical grinding, chipping, laser cutting, etc., are usually more effective when the processed material is dry and hard, as compared with a moist mixture. Therefore, the separated solid phase material may be subjected to at least one such trimming or breaking-up method after being at least partially dewatered. The trimming or breaking-up of the solid material may produce smaller solid particles which may be reintroduced into the liquid phase of the waste material, for continued processing.

The solid-liquid separation of step 114 may be utilized simultaneously with any one or more of the steps of process 100, and/or prior to or subsequently to any one or more of the steps. As a non-limiting example, the waste material may undergo solid-liquid separation prior to saccharification. Alternatively, solid-liquid separation may be performed as a pre-step to maintaining the waste material under conditions that inhibit microbial activity (i.e., step 104), where the resulting liquid phase may be put under the conditions that inhibit microbial activity while the solid phase may undergo breaking-up treatment as described above, after which the degraded solid phase is added back into the liquid phase. Further alternatively or additionally, the solid-liquid separation may be performed prior to fermentation (i.e., step 110).

In some embodiments, following a solid-liquid separation, the liquid phase is concentrated to obtain a concentrated liquid or syrup containing the reducing sugars. In some embodiments, the concentrated liquid or syrup containing the reducing sugars is produced in a first location, and is subsequently transported to a second location in which the production of lactic acid from the concentrated liquid or syrup is conducted. In some embodiments, the first location is an organic waste collection facility and the second location is a lactic acid production facility. In some embodiments, sterilization of the concentrated liquid or syrup is performed at the second location, prior to lactic acid production from the concentrated liquid or syrup by fermentation. In some embodiments, the concentrated liquid or syrup is maintained under the conditions disclosed herein during the transportation.

As used herein and in the appended claims, the term “about” refers to ±10%, for example +/−5%, +/−1%, and +/−0.1% from the specified value.

As used herein and in the appended claims, the singular forms “a”, “an”, and “the” include plural references unless the context clearly dictates otherwise. Thus, for example, reference to “an enzyme” includes a plurality of enzymes and types of enzymes, unless the context clearly dictates otherwise. It should be noted that the term “and” or the term “or” are generally employed in its sense including “and/or” unless the context clearly dictates otherwise.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Inhibition of Endogenous Microbial Activity Within an Organic Waste Material

The following experiment was performed in order to demonstrate the efficacy of maintaining an organic waste at the temperature and pH conditions of the present invention (e.g., at a temperature between 55° C. to 65° C. and pH between 3.5 to 5.5) in inhibiting endogenous microbial activity.

### Procedure:

A non-sterile slurry of mixed food waste collected from supermarket logistic returns and surplus was provided. The mixed food waste contained expired bakery and dairy products, beverages, and fruits and vegetables, and was subjected to separation of inorganic solid components (plastics, etc.) and griding. The slurried waste contained 60 g/l of potential glucose and a concentration of 17 g/l of available glucose. Potential glucose, interchangeably also referred to as “glucose potential”, indicates the concentration of both sugars that are in a non-reducing state before saccharification, and fermentable sugars which are in a reducing state, i.e., available for fermentation (“available glucose”).

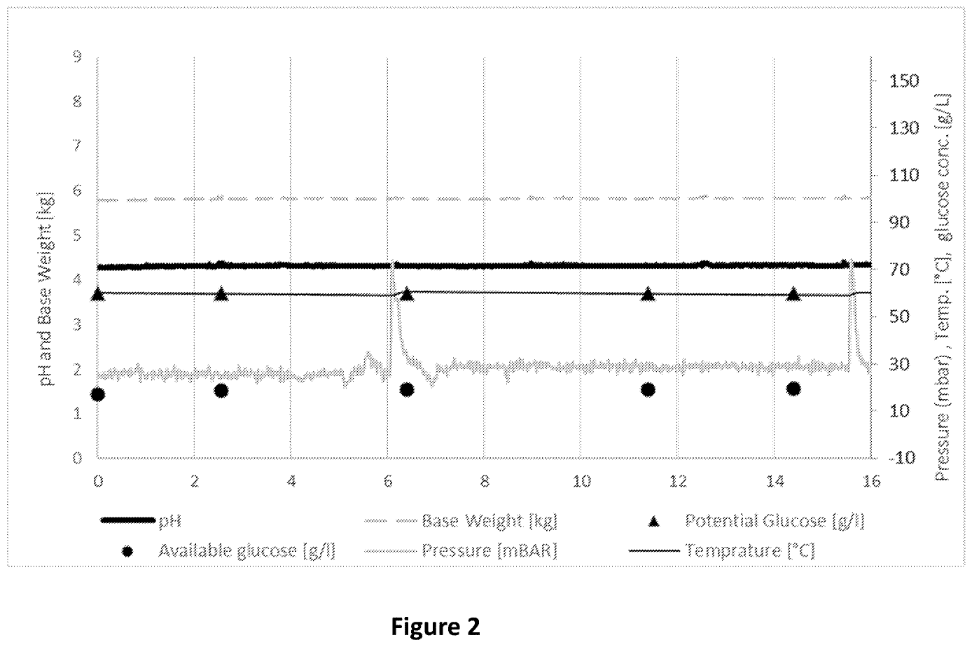

The waste material was pumped into a bioreactor heated to a temperature of 60° C. (thin black line in FIG. 2). The pH of the waste material was 4.3-4.4, and thus no pH adjustment was performed. The waste material was maintained in the heated bioreactor for 16 h. The pH, base weight [kg], pressure (mbar), potential glucose concentration and available glucose concentration (g/l) were measured intermittently by calibrated instruments during the 16 hours.

### Results:

The results are shown in FIG. 2.

The thick black line indicates pH. The pH remained steady between 4-5, validated with offline measurements at pH 4.3-4.4. Since glucose consumption and subsequent production of acids is indicative of microbial activity, the substantially constant pH indicates that no microbial activity occurred during the experiment. Accordingly, no base was added to the waste material (dashed grey line). The suppression of microbial activity is also evident from the pressure measurements (thick grey line) which remained steady with no production of gasses, e.g., CO₂, methane.

Black triangles (▴) indicate glucose potential. Black dots (●) indicate the concentration of available fermentable reducing sugar only, e.g., glucose. The initial glucose potential and concentration of available glucose, of 60 g/l and 17 g/l respectively, were maintained throughout the 16 hours of the experiment, indicating that no microbial activity involving utilizing free glucose occurred.

When a non-sterile mixed food waste slurry is not maintained under the conditions of the present invention (e.g., kept at room temperature), significant gas production is observed, indicating unwanted microbial activity within the waste. The above results show that by maintaining a non-sterile organic waste slurry under the conditions according to the present invention, e.g., at a temperature of 60° C. and at a pH of pH 4-5, microbial activity, including glucose consumption and gas production, is inhibited.

### Example 2

### Heat Treatment Followed by Enhanced Saccharification

The following experiment was performed in order to test the effect of a heat treatment on subsequent saccharification.

### Procedure:

A non-sterile slurry of mixed food waste material as in Example 1 was provided, which contained a glucose potential of 60 g/l, and available glucose concentration of 17 g/l.

The waste material was pumped into a bioreactor. A first dose of saccharification enzymes (glucoamylase (GA) and alpha-amylase (AA)) was added, after which the temperature was heated to 60° C. The pH of the waste material was 4.3-4.4, and thus no pH adjustment was performed. The waste material was maintained at 60° C. for 18 h with stirring. After 18 hours, the temperature was steadily increased, reaching 70° C. and remaining in the range of 70-73° C. for 2 hours. After cooling down back to 60° C., at 22 h, a second dose of the saccharification enzymes was added and the temperature was maintained at 60° C. for additional 2.5 hours.

The pH, pressure (mbar), potential glucose concentration and available glucose concentration (g/l) were measured by a calibrated instruments intermittently during the process.

### Results:

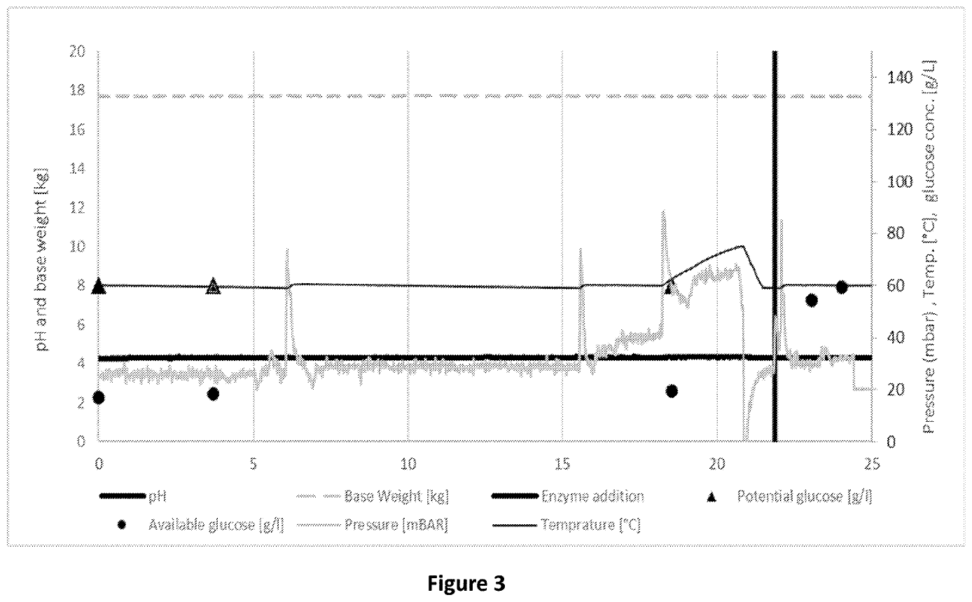

The results are shown in FIG. 3.

The thick black line indicates pH. The pH remained between 4-5, validated with offline measurements at pH 4.3-4.4. Since glucose consumption and subsequent production of acids is indicative of microbial activity, the substantially constant pH shows that no microbial activity occurred during the experiment. Accordingly, no base was added to the waste material (dashed grey line).

The vertical black bar at 22 h indicates enzyme addition after the 70° C. heat treatment (temperature indicated by thin black line). Two types of enzymes were added to induce the saccharification, glucoamylase (GA) and alpha-amylase (AA).

Black triangles (▴) indicate glucose potential. Glucose potential indicates the concentration of both sugars that are in a non-reducing state (before saccharification) and fermentable sugars which are in a reducing state, i.e., available for fermentation. Black dots (●) indicate the concentration of available fermentable reducing sugar only, e.g., glucose. The waste contained 60 g/l of potential glucose, of which 17 g/l constitute available glucose. After addition of the first dose of saccharification enzymes and 18 hours at 60° C. and pH 4.4, no notable change in available glucose was measured. Following the heat treatment and addition of a second dose of saccharification enzymes, substantially all the potential glucose was converted into available glucose (59.5 g/l).

The thick grey line indicates pressure measurement (mbar). It is noteworthy that although the pressure began to increase at 18h from about 40 mbar to about 65 mbar, this increase in pressure was the result of the increase in temperature, which was initiated at the same time, from 60° C. to over 70° C. The elevated pressure was not a result of gas production due to microbial utilization of available glucose, as the steady pH and glucose levels clearly indicate that there was no such microbial activity. This is evident also by the drop in pressure beginning at about 21 h, corresponding to a concurrent cooling down of the waste material.

Taken together, these results show that subjecting a non-sterile organic waste material to a heat treatment according to the present invention (e.g., at a temperature of 70° C. for 2 hours as performed in the present example) prior to saccharification substantially increases the efficiency of the saccharification and thus the yield of reducing sugar available for fermentation.

Saccharification of polysaccharides for fermentation processes is typically carried out on a polysaccharide-containing substrate after it was subjected to sterilization. An organic waste slurry, particularly a food waste slurry, at its raw condition includes various solids, which make it difficult to effectively sterilize the waste, and even if sterilized, it is difficult to maintain the waste under sterile conditions throughout its processing until the lactic acid production stage. The foregoing experiment demonstrates that a heat treatment according to the present invention enables performing efficient saccharification on a non-sterile organic waste slurry, thus improving the process of organic waste pretreatment.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the spirit and scope of the present invention as described by the claims, which follow.

1. A method for pretreating organic waste prior to large-scale production of lactic acid or a salt thereof from the organic waste, the method comprising:
(a) providing a non-sterile slurry of organic waste comprising non-reducing polysaccharides and reducing sugars;
(b) maintaining the non-sterile slurry of organic waste of step (a) at a temperature in the range of 55° C. to 65° C. and at a pH in the range of 3.5 to 5.5 for a first time duration of at least 1 hour, to thereby suppress endogenous microbial activity in the organic waste;
(c) raising the temperature of the non-sterile slurry of organic waste of step (b) to a temperature above the temperature of step (b) and between 65° C. to 85°° C. for a second time duration of at least 0.5 hour;
(d) optionally adjusting the temperature to a temperature between 45° C. to 65° C.; and
(e) adding one or more saccharifying enzymes to the non-sterile slurry of organic waste of step (c) or step (d) for hydrolyzing polysaccharides in the organic waste to release reducing sugars, to obtain saccharified organic waste.

2. The method of claim 1, wherein step (c) and step (e) are performed simultaneously.

3. The method of claim 1, wherein the organic waste comprises plastics and/or inorganic solid components and the method comprises subjecting the organic waste to separation of said plastics and/or inorganic solid components prior to step (a).

4. The method of claim 1, further comprising separating the saccharified organic waste into a liquid phase comprising reducing sugars and a solid phase, and subjecting the liquid phase to sterilization.

5. The method of claim 1, wherein step (d) is performed and wherein the method further comprises
separating the saccharified organic waste into a liquid phase comprising reducing sugars and a solid phase, and subjecting the liquid phase to sterilization,
thereby pretreating the organic waste to obtain a feedstock material for large-scale production of lactic acid or a salt thereof.

6. The method of claim 1, wherein the one or more saccharifying enzymes comprise at least one of: a glucoamylase, an alpha-amylase and a pullulanase.

7. The method of claim 1, wherein the second time duration of step (c) is in the range of 0.5 hours to 5 hours.

8. A method for pretreating organic waste prior to large-scale production of lactic acid or a salt thereof from the organic waste, the method comprising:
(A) providing a non-sterile slurry of organic waste comprising non-reducing polysaccharides and reducing sugars;
(B) heating the non-sterile slurry of organic waste to a temperature between 65° C. to 85°° C. for a time duration of at least 1 hour;
(C) optionally adjusting the temperature to a temperature between 45° C. to 65° C.; and
(D) adding one or more saccharifying enzymes to the non-sterile slurry of organic waste of step (B) or step (C) for hydrolyzing polysaccharides in the organic waste to release reducing sugars, to obtain saccharified organic waste.

9. A method for suppressing endogenous microbial activity in an organic waste material prior to subjecting the waste material to controlled large-scale production of lactic acid or a salt thereof, the method comprising the steps of:
(a) providing a non-sterile slurry of organic waste material comprising at least one of non-reducing polysaccharides and reducing sugars; and
(b) maintaining the waste material of step (a) at a temperature in the range of 55° C. to 65° C. and at a pH in the range of 3.5 to 5.5 for a first time duration of at least 1 hour, to thereby suppress endogenous microbial activity in the waste material prior to subjecting the waste material to controlled large-scale production of lactic acid or a salt thereof.

10. The method of claim 9, wherein the organic waste comprises plastics and/or inorganic solid components and the method comprises subjecting the organic waste to separation of said plastics and/or inorganic solid components prior to step (a).

11. The method of claim 1, wherein step (b) is performed at a pH in the range of 4 to 5.

12. The method of claim 1, wherein the first time duration of step (b) is in the range of 1 to 96 hours.

13. The method of claim 1, wherein the first time duration of step (b) is in the range of 12 to 48 hours.

14. The method of claim 1, further comprising applying mechanical treatment to the waste material prior to, simultaneously with, and/or subsequently to step (b).

15. The method of claim 1, wherein the waste material is selected from the group consisting of food waste, municipal waste, agricultural waste, plant material, and a combination thereof.

16. The method of claim 1, wherein the non-sterile slurry of organic waste contains microbes at a concentration of at least 10⁵ CFU/ml.

17. The method of claim 1, wherein the non-sterile slurry of organic waste contains microbes at a concentration of at least 10⁷ CFU/ml.

18. A method for producing lactic acid from organic waste, the method comprising the steps of:
(i) subjecting the organic waste to pretreatment comprising a pretreatment method according to claim 1; and
(ii) adding a lactic acid producing microorganism to the pretreated organic waste and incubating in a fermentation reactor under controlled conditions for lactic acid production by the lactic acid producing microorganism, to thereby produce lactic acid.
